(19) United States
(12) Patent Application Publication
(10) Pub. No.: US 2024/0282430 A1
(43) Pub. Date: Aug. 22, 2024

(54) SCORING AND CLASSIFYING EXTRACTS FROM A NON-ANIMAL NATURAL EUKARYOTIC SOURCE

(71) Applicant: SEEKWELL LTD., Tel Aviv (IL)

(72) Inventors: Oded SHOSEYOV, Shoham (IL); Ronit SHALTIEL-KARYO, Ramat Hasharon (IL); Ron SHIMONI, Hod Hasharon (IL); Eden FIRUZ, Netiv Haasara (IL)

(21) Appl. No.: 18/570,279

(22) PCT Filed: Jun. 14, 2022
(86) PCT No.: PCT/IL2022/050638

§ 371 Date: Dec. 14, 2023

Related U.S. Application Data
(60) Provisional application No. 63/202,497, filed on Jun. 14, 2021.

(51) Int. Cl. G16H 20/90

(52) U.S. Cl. CPC G16H 20/90

(57) ABSTRACT

There is provided a technique of classifying medical plant and/or mushroom extracts. The technique comprises: for a sample of a medical plant and/or mushroom extract to be classified, obtaining results of a set of bioassays informative of modulating properties of the sample, thereby generating a bioassay data informative of values of attributes related to the modulating properties; applying to the bioassay data a trained machine learning model (MLM) to classify the sample into a class of one or more predefined classes, wherein the MLM is trained to classify samples into the one or more predefined classes in accordance with bioassay data thereof, the training provided with the help of a training set comprising a plurality of training samples, each characterized by a respective bioassay data and a class associated with the sample. The technique is applicable, for example, for standardization of the extracts in accordance with the intended medical use.

### TECHNICAL FIELD

The presently disclosed subject matter relates to industrial use of products based on medical plants and/or fungi and, more particularly, to standardization of such products.

### BACKGROUND

Medical extracts based on extracts obtained from non-animal eukaryotic sources, such as plants and fungi (or mushrooms) have been used in many different cultures for thousands of years for treatment of various ailments and conditions. Such medical extracts can be implemented, for example, in cosmetic products, over-the-counter health products, medical devices, pharmaceutical drugs, etc.

It is noted that the term “extract” refers to any concentrated preparation or solution of a compound or drug derived from a non-animal eukaryotic natural source, which includes one or more plants and/or fungi (mushrooms). Extracts may be prepared by any suitable process known in the art (e.g. steeping a herb in solution, drying and grinding a herb into a powder, dissolving the powder in a solution, etc.). An extract may be further concentrated by removing a portion of the solvent after dissolving an amount of the desired compound in the solution. An extract may also be strained or centrifuged to remove any solid material from the solution. The term “extract” should be expansively construed to include single extracts and/or combinations thereof.

A composition of an extract from a non-animal eukaryotic source can vary depending on growing conditions, genetic variation, the timing of planting and harvesting, the age of the plant matter when the extract is prepared, method of extraction etc. Unlike controllable synthetic medicinal compositions, in vivo activity of extracts from different batches of the same non-animal eukaryotic source can vary. Moreover, different batches with different in vivo activity can share very similar chemical profiles making standardization a very difficult task.

Problems of standardization of extracts from non-animal eukaryotic sources for the treatment of diseases have been recognized in the conventional art and various techniques have been developed to provide solutions, for example: US Patent Publication No. 2015/021961 entitled “Integrated Systems and Methods of Evaluating Cannabis And Cannabinoid Products for Public Safety, Quality Control and Quality Assurance Purposes” discloses embodiments facilitating suppliers and consumers of a Cannabis and/or cannabinoid product to evaluate the origin, efficacy, potency, and quality of the product. Embodiments of the invention also include Cannabis processing center where samples of the products are analyzed for research studies to test and set parameters for third parties. Embodiments of the invention include, for example, analyzing Cannabis products to determine quality and quantity of desired components and undesired components, determining concentrations of cannabinoids in the product, and comparing measures of components in the product against regulations.

US Patent Publication No. 2020/0126633 entitled “Method for Controlling the Quality of Traditional Chinese Patent Medicines Based on Metagenomics” discloses a method including extracting genomic DNA of a sample of the traditional Chinese patent medicine, constructing a library of the genomic DNA based on a high-throughput sequencing platform, and performing metagenomic sequencing. The data obtained from the metagenomic sequencing is processed to obtain the ITS2 sequence of the traditional Chinese patent medicine sample A BLAST alignment is performed on the ITS2 sequence in the DNA Barcoding System for Identifying Herbal Medicine, to obtain species identification results. The obtained identification results are compared with the labeled species of the traditional Chinese patent medicine to obtain a conclusion about the quality of the traditional Chinese patent medicine sample

US Patent Publication No. 2019/0374593 entitled “Mechanism Based Quality Control for Botanical Medicine” discloses methods of evaluating the quality of a batch of a herbal composition, the method comprising subjecting a test batch of the herbal composition to one or more biological analysis methods and comparing the results derived from the test batch to the results of a known batch of herbal composition which has a known in vivo effect.

US Patent Publication No. 2021/0055276 entitled “Detection Method for Quality Grade Of Traditional Chinese Medicine” discloses a detection method including: detecting the levels of quality control index components of Traditional Chinese Medicine (TCM) and efficacy-related in vitro activity by establishing a correlation between principal components of TCM and in vitro activity; determining the state of sample cluster by principal component analysis; constructing a logistic regression model of quality grade versus index components and bioactivity and establishing corresponding grade detection formulas of Chinese medicinal materials by fitting a large number of sample data for Chinese medicinal materials from different places of origin and batches.

The references cited above teach background information that may be applicable to the presently disclosed subject matter. Therefore, the full contents of these publications are incorporated by reference herein where appropriate for appropriate teachings of additional or alternative details, features and/or technical background.

### GENERAL DESCRIPTION

For purpose of illustration only, the following description is provided for Cannabis-based products. It is noted that the teachings of the presently disclosed subject matter are not bound by Cannabis and, likewise, applicable to other non-animal eukaryotic sources including medical plants and/or mushrooms (e.g. psilocybin mushrooms, vanilla plants, etc.).

The therapeutic potential of Cannabis and Hemp spans over a wide spectrum, e.g. anti-inflammatory, pain relief, skeletal muscle relaxation, anti-microbial indications, neuronal protection, etc. It is a common knowledge that Cannabis contains hundreds of molecules that effect its therapeutic activity, due to the well documented “entourage effect”, wherein certain combinations of active ingredients are more effective as anti-inflammatory, while others may be better suited as anti-microbial. The common method to address the variability of the plant and predict therapeutic suitability is the chemical approach, according to which a Cannabis product or an extract is prescribed based on its chemical composition, e.g., typically based on the relative amounts of CBD, THC and in some instances even CBG and CBC, and not based on its actual biological or pharmacological effectiveness.

As a may be known, the interplay between hundreds of different compounds found in a Cannabis extract, e.g., hemp oil or powder, is huge. Even in cases where two or more Cannabis extracts exhibit substantially identical chemical profiles (major cannabinoids), the biological effects demonstrated by each may completely differ due to a great number of varying factors, including, inter alia, presence or absence of minute amounts of minor cannabinoids or other chemicals such as terpenes or flavonoids that can have an effect on the overall effect of the extract; the Cannabis strain used; the method used for obtaining the extract; the growing season; the growing conditions; the source of the extract; and others.

The inventors have recognized and appreciated that a great percentage of chemical-based matching and/or predictions do not actually mature into actual therapeutic effectiveness in vivo, as the chemical approach fails to appropriately correlate between an extract composition, e.g., presence of specific cannabinoids, and a therapeutic effect.

Based on intensive research, the inventors have defined strong correlation between values of bioassay results and Cannabis extracts best usable for different fields of treatment (e.g. treatment of inflammation, oxidative stress, pain, itch, cognition, etc.). The fields of treatment are referred to hereinafter as “classes”.

In accordance with certain embodiments of the presently disclosed subject matter, there is proposed a technique of predicting a biological effect of a new plant and/or fungi extract and respective classification using pre-defined in vitro biochemical assays. The technique allows to classify the new extract utilizing a classifier trained on a large number of previously tested extracts.

Among advantages of certain embodiments of the presently disclosed subject matter is capability to predict an expected therapeutic effect whilst substantially diminishing variability derived from extracts produced via different extraction methods or extracts derived from different Cannabis strains or extracts derived from different sources.

Likewise, the provided method can enable screening a plurality of extracts to reveal the extracts suitable for manufacturing products devoted to a certain treatment, i.e. belonging to a given class and/or predicting a score indicative of usability of the sample (and respective extract) for a given class (i.e. efficacy for corresponding treatment).

In certain embodiments, the extract can be further selected for further use when results of provided one or more bioassays are within a pre-defined efficacy bar. The efficacy bar is a qualifying score (or range of scores) informative of the target level of efficacy in a certain biological assay when provided for a class of interest. By way of non-limiting example for inflammation treatment, in bioassay of COX2 inhibition the target level of efficacy (efficacy bar) can be set as 80% of enzyme's inhibition, in bioassay of IL-6 production the target level of efficacy can be set as 50% inhibition, etc. It is noted that the value of efficacy bar can be further modified in accordance with data aggregated for a certain bioassay during certain time frame.

In accordance with certain embodiments of the presently disclosed subject matter, there is proposed a method of computer-based classifying a medical extract from a non-animal eukaryotic source. The method comprises: for a sample of a non-mammalian eukaryotic extract to be classified, obtaining results of a set of one or more bioassays informative of modulating properties of the sample, thereby generating a bioassay data informative of values of attributes related to the modulating properties; and applying to the bioassay data a trained machine learning model (MLM) to classify the sample into a class of one or more predefined classes. The MLM is trained to classify samples into the one or more predefined classes in accordance with bioassay data thereof, the training provided with the help of a training set comprising a plurality of training samples, each characterized by a respective bioassay data and a class associated with the sample.

When the classifying is provided to define belonging the sample to a given class, the method further comprising predefining a set of bioassays and respective attributes related to the given class.

The extracts can be classified in accordance with the class of the respective samples. The classified extracts can be used, for example, for manufacturing a medical product with treatment properties corresponding to the respective class, standardization of medical extracts in accordance with the one or more classes, screening the extracts to reveal one or more extracts belonging to a given class, etc.

In accordance with further aspects and, optionally, in combination with other aspects of the presently disclosed subject matter, the method can further comprise applying to the bioassay data a trained machine learning model (MLM) to obtain a score indicative of efficacy of the sample for the class it has been classified, wherein the MLM can be trained to score samples for respective class in accordance with bioassay data thereof, the training can be provided with the help of a training set comprising a plurality of training samples, each characterized by a respective bioassay data and a score for the respective class.

In accordance with further aspects and, optionally, in combination with other aspects of the presently disclosed subject matter, generating the training set can comprise providing the one or more bioassays to a plurality of training samples to yield a feature space representing the obtained bioassay data; and applying predefined rules to the feature space to select, among the plurality of training samples, training samples to be included in the training set.

The method can further comprise clustering the samples in the feature space prior to selecting the training samples for the training set, wherein the predefined rules specify, at least, the minimal and/or the maximal numbers of selected training samples in the training set, the rules of selection the samples between the clusters and the rules of selection within the clusters.

The predefined rules can further consider ranking the relevance of bioassays and/or attributes thereof for the respective classes.

In accordance with further aspects and, optionally, in combination with other aspects of the presently disclosed subject matter, there is provided a computer-based method of providing a medical extract from a non-animal eukaryotic source with a score indicative of efficacy of the extract for a given class. At a setup phase, the method comprises: upon specifying one or more bioassays relevant for the given class, a) providing for a plurality of training samples a bioassay among the one or more bioassays to obtain bioassay results informative of modulating properties of the respective samples; repeating operation a) for all the one or more bioassays thereby obtaining bioassay data for the training samples from the plurality of training samples; generating a training set comprising, for each training sample included therein data informative of its bioassay data and a respective sample's score. The method further comprises using the training set to obtain a machine-learning model trained to score samples in accordance with bioassay data thereof. At a runtime phase, the method comprises providing the one or more bioassays for a new sample, thereby generating bioassay data informative of modulating properties thereof; and applying the trained machine learning model to its bioassay data to provide a score of the new sample with regard to the given class.

In accordance with other aspects of the presently disclosed subject matter, there is provided a set of bioassays usable for classifying a medical extract from a non-animal eukaryotic source in accordance with any aspects of the methods above.

In accordance with other aspects of the presently disclosed subject matter, there is provided a set of bioassays usable for provide a score for a medical extract from a non-animal eukaryotic source in accordance with any aspects of the methods above.

In accordance with other aspects of the presently disclosed subject matter, there are provided one or more computing devices comprising processors and memory, the one or more computing devices configured, via computer-executable instructions, to perform operations for operating, in a cloud computing environment, in accordance with any aspects of the methods above.

In accordance with other aspects of the presently disclosed subject matter, there is provided a classification system for classifying and/or scoring a medical extract in accordance with any aspects of the methods above.

In accordance with other aspects of the presently disclosed subject matter, there is provided a non-transitory computer-readable medium comprising instructions that, when executed by a computing system comprising a memory storing a plurality of program components executable by the computing system, cause the computing system to operate in accordance with aspects of the methods above.

### BRIEF DESCRIPTION OF THE DRAWINGS

In order to understand the invention and to see how it can be carried out in practice, embodiments will be described, by way of non-limiting examples, with reference to the accompanying drawings, in which:

FIG. 1 illustrates a block diagram of a classification system in accordance with certain embodiments of the presently disclosed subject matter;

FIG. 2 illustrates a schematic representation of mapping a set of samples of Cannabis extracts to a multi-dimensional feature space in accordance with certain embodiments of the presently disclosed subject matter; and

FIG. 3 illustrates a generalized flow-chart of classifying a sample of medical plant and/or mushroom extract in accordance with certain embodiments of the presently disclosed subject matter;

FIG. 4 illustrates a generalized flow-chart of scoring a sample of medical plant and/or mushroom extract in accordance with certain embodiments of the presently disclosed subject matter;

FIG. 5a illustrates a schematic diagram of “smart sampling” training samples of medical plant extract in accordance with certain embodiments of the presently disclosed subject matter; and

FIG. 5b illustrates a generalized flow-chart of “smart sampling” training samples of medical plant extract in accordance with certain embodiments of the presently disclosed subject matter

### DETAILED DESCRIPTION

In the following detailed description, numerous specific details are set forth in order to provide a thorough understanding of the invention. However, it will be understood by those skilled in the art that the presently disclosed subject matter may be practiced without these specific details. In other instances, well-known methods, procedures, components and circuits have not been described in detail so as not to obscure the presently disclosed subject matter.

Unless specifically stated otherwise, as apparent from the following discussions, it is appreciated that throughout the specification discussions utilizing terms such as “processing”, “computing”, “applying”, “selecting”, “generating”, “assigning” or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities including, by way of non-limiting example, the classifier and processing circuitry therein disclosed in the present application.

The operations in accordance with the teachings herein may be performed by a computer specially constructed for the desired purposes or by a general-purpose computer specially configured for the desired purpose by a computer program stored in a non-transitory computer-readable storage medium.

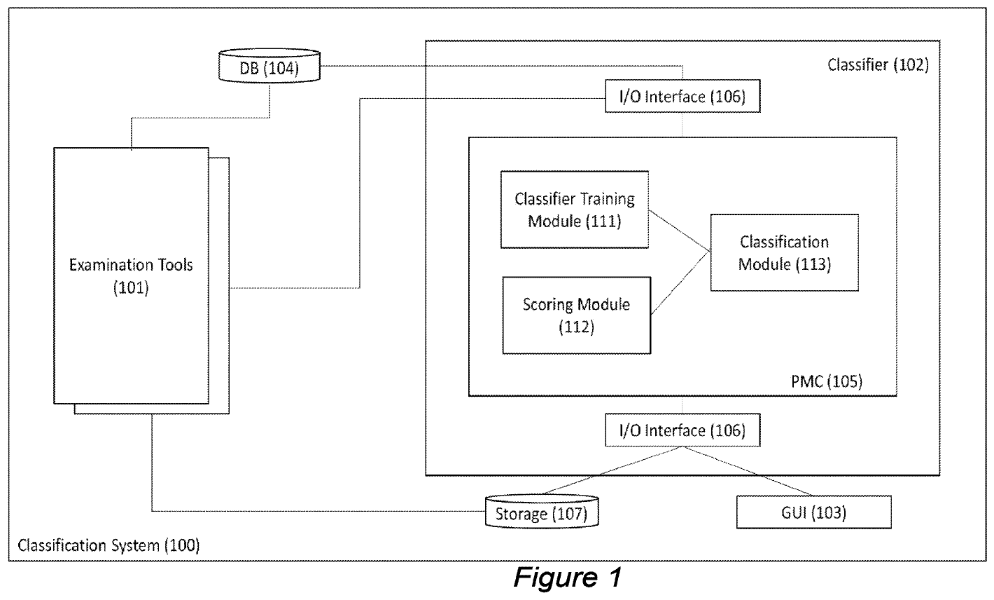

Bearing this in mind, attention is drawn to FIG. 1 illustrating a generalized block diagram of a classification system 100 usable for classifying Cannabis extracts in accordance with certain embodiments of the presently disclosed subject matter.

Classification of the extracts can be provided by classifying samples thereof (referred to hereinafter also as samples), and classification of a given extract corresponds to classification of its sample(s). Classification system 100 can be used for classification of samples as a part of Cannabis-based products fabrication; the classification can be carried out before, during or after manufacturing of such products.

The classification system can comprise a variety of examination tools 101. The term “examination tool(s)” used herein should be expansively construed to cover any tool usable for bioassay informative of modulating (e.g. inhibiting and/or elevating) its properties by a sample. Bioassays enabled by examination tools 101 are configured to examine the sample in order to provide values of one or more attributes indicative of the modulating properties. Non-limiting examples of the bioassays and respective attributes are further detailed below.

As used herein, the term “modulating properties” or any lingual variation thereof refers to the capability of a tested sample, e.g., extract, to inhibit or activate or increase or demonstrate a change from a threshold value indicative or characteristic of a certain bioassay or a combination of such assays. The modulation may be of any degree or size relative to a control, which may be a comparative extract of known properties, or a control known and acceptable in the art.

It is noted that the teachings of the presently disclosed subject matter are not bound by classes, bioassays and attributes disclosed herein and, likewise, applicable to other classes and/or other bioassays informative of modulating (e.g. inhibiting and/or elevating) the same or other predefined properties of the samples.

Classification system 100 further comprises classifying tool (referred to hereinafter also as a classifier) 102 configured to automatically classify the samples into a plurality of classes. Classification may have different purposes, and the classification results can be used, for example, for screening and/or scoring Cannabis extracts to identify the most suitable for manufacturing a certain product, sorting Cannabis extracts in accordance with predefined criteria, standardize the extracts for further manufacturing and/or trading, etc.

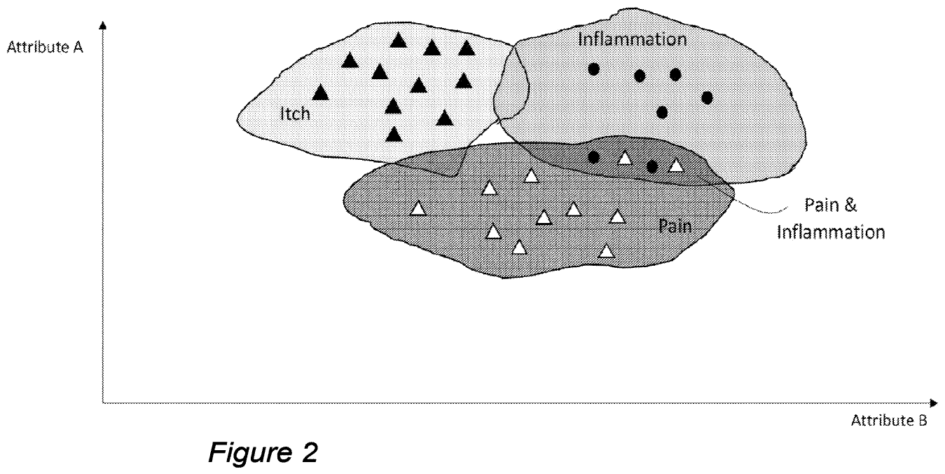

Classifier 102 comprises a processor and memory circuitry (PMC) 105 operatively connected to a hardware-based input/output interface 106. PMC 105 is configured to provide processing necessary for operating the classifier as further detailed with reference to FIGS. 2-4. PMC 105 comprises a processor and a memory (not shown separately within PMC 105). Operation of classifier 102 and PMC 105 therein will be further detailed with reference to FIGS. 2-4.

The processor of PMC 105 can be configured to execute several program components in accordance with computer-readable instructions implemented on a non-transitory computer-readable memory comprised in the PMC. Such executable program components are referred to hereinafter as functional modules comprised in the PMC 105.

Functional modules comprised in the PMC 105 can include classifier training module 111, scoring module 112 and classification module 113.

For purpose of illustration only, in the following description classifier 102 is exemplified as multi-class or single-class classifier operating in accordance with one or more classification rules. It is noted that the teachings of the presently disclosed subject matter are not bound by the architecture of classifier 102.

The classification rules specify a classification engine (e.g. support vector machine (SVM), random forest classification engine, neural networks, etc.) and a plurality of confidence thresholds which can differ for different classes. Further to classifying the samples into the classes, the classifier can be configured to define, for each given sample, a confidence level indicative of probability that the sample belongs to a certain class, and to assign the given sample to the certain class if the confidence level meets the respective confidence thresholds.

Classifier 102 is operatively connected to one or more examination tools 101, Graphical User Interface (GUI) 103, data repository 104 and storage unit 107. Bioassay data of the samples (i.e. data informative of the modulation results of bioassays and/or derivatives thereof and/or metadata associated therewith) can be transmitted from the examination tools 101 to the classifier 102 via GUI 103 and/or input/output interface 106. Classification results may be further transmitted to data repository 104 and/or storage unit 107. GUI 103 is configured to enable user-specified inputs and render outputs related to system 100.

Storage unit 107 and/or data repository 104 can be configured to store any data necessary for operating system 100, e.g., data related to input and output of system 100, as well as intermediate processing results generated by system 100. In addition, the storage unit 107 and/or data repository 104 can be configured to store data including bioassay data produced by the examination tool(s). Accordingly, the data can be retrieved therefrom and provided to the PMC 105 for further processing. Additionally or alternatively, system 100 can be operatively connected to one or more external data repositories (not shown in FIG. 1) which are configured to store data required by system 100 (e.g. one or more training sets).

Those skilled in the art will readily appreciate that the teachings of the presently disclosed subject matter are not bound by the system illustrated in FIG. 1; equivalent and/or modified functionality can be consolidated or divided in another manner and can be implemented in any appropriate combination of software with firmware and hardware. At least part of the functionality of the classification system can be implemented in a cloud and/or distributed and/or virtualized computing arrangement.

It is further noted that in other embodiments at least part of the examination tools 101, data repository 104, GUI 103 can be external to the classification system 100 and operate in data communication with classifier 102 via input/output interface 106.

Optionally, classifier 102 can be implemented as one or more stand-alone computers usable in conjunction with one or more examination tools. Alternatively, at least part of the functions of classifier 102 can be integrated with one or more examination tools. Optionally, classifier 105 can operate on pre-acquired bioassay results characterizing a given sample and stored in storage unit 107 and/or data repository 104 and/or received via input/output interface 106 from an external source via a communication network.

As disclosed above, a sample can be characterized by values of attributes informative of modulating properties of the sample defined in one or more bioassays from a predefined set of bioassays. Data informative of the modulation results and/or derivatives thereof and/or metadata associated therewith are referred to hereinafter as “bioassay data”. By way of non-limiting example, bioassay data of a given sample can be represented by a multi-dimensional vector with respective attributes' values. A set of attributes for a given bioassay can be predefined or can be derived by processing bioassay results for a plurality of samples (e.g. with the help of appropriate machine-learning model).

Referring to FIG. 2, there is illustrated a schematic representation of a multi-dimensional feature space to which a set of samples is mapped. As will be further detailed below by way of non-limiting example, the samples can be characterized by values of attributes received in a predefined set of bioassays including at least one of the Cytokine screening: IL-6; Cytokine screening: TNFa; ROS expression; Cox2 inhibition; plaque formation/dissolution; ratio of reduced to oxidized glutathione; TRPV1 agonist; inhibition of acetylcholine esterase activity; Neuroprotection and others.

For the sake of visual simplicity, the feature space is represented in FIG. 2 as being two-dimensional, but the classification processes that are described herein can be carried out in spaces of higher dimensionality. As illustrated by way of non-limiting example in FIG. 2, in accordance with their attribute values, the samples can belong to different predefined classes. Namely, the samples denoted by black triangles belong to the class “Itch treatment”, denoted by white triangles belong to the class “Pain treatment” and the samples denoted by circles belong to the class “Inflammation treatment”. As the illustrated classes are overlapped, it is possible that a sample belongs to two or more classes. Alternatively, it is possible to define combined classes—for example some of the illustrated samples match a combined class of “Pain and Inflammation treatment”. The borders and shapes of the classes depend on the confidence levels defined in the classification rules.

In embodiments of a multi-class classification, a given sample can be classified into one or more classes of a plurality of predefined classes. In embodiments of a single-class classification, a given sample can be classified in according with belonging/not belonging to a class of interest. Additionally or alternatively, a sample can be associated with a score indicative of usability (efficacy) of the sample for a certain class.

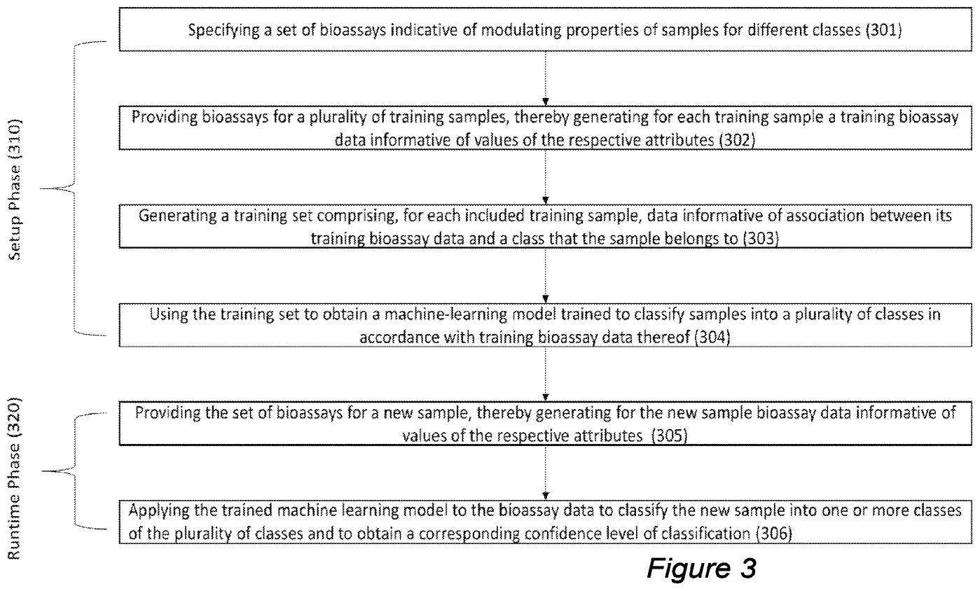

Referring to FIG. 3, there is illustrated a generalized flow-chart of machine learning-based classification of samples into one or more classes in accordance with certain embodiments of the presently disclosed subject matter. The process includes a setup phase 310 of training the classifier to provide classification of the samples and runtime stage 320 of providing classification of each given new sample.

The setup phase can comprise specifying (301) a set of bioassays indicative of modulating (e.g. inhibiting and/or elevating) properties of samples and providing bioassays for a plurality of training samples, thereby generating (302) for each training sample bioassay data (e.g. a multi-label vector) informative of values of bioassay results.

Optionally, bioassay can be informative of various modulating properties, while bioassay data can include and thereby be informative merely of values of one or more attributes defined as “significant” for each of provided bioassays. A set of attributes significant for a given bioassay can be predefined by an expert or can be derived from bioassay results with the help of statistical and/or mathematical manipulations (e.g. by applying a respectively trained machine learning model).

The setup phase can further comprise generating (303) a training set comprising, for each training sample therein, data informative of association between its bioassay data and a class that the sample belongs to; and using the training set to obtain (304) a machine-learning model trained (e.g. by classifier training module 111) to classify samples into a plurality of classes in accordance with bioassay data thereof. A class of a training sample can be defined with involvement of an expert. The runtime phase can comprise: providing the set of bioassays for a given sample to be classified, thereby generating bioassay data informative of values of the respective attributes (305); applying (306) the trained machine learning model (e.g. by classification module 113) to the bioassay data to classify the given sample into one or more classes of the plurality of classes and, optionally, to obtain a corresponding confidence level of classification.

Optionally, the above method can be applied for screening a plurality of extracts to reveal the extracts suitable for manufacturing products devoted to a certain treatment, i.e. belonging to a given class. In such a case of a single-class classification, the set of bioassays and/or predefined attributes can be limited by the bioassays and/or attributes related to the given class, and the samples can be classified in according to belonging/not belonging to the class of interest.

Alternatively or additionally, the machine learning model can be trained to predict a score indicative of usability of the sample (and respective extract) for a given class (i.e. efficacy for corresponding treatment).

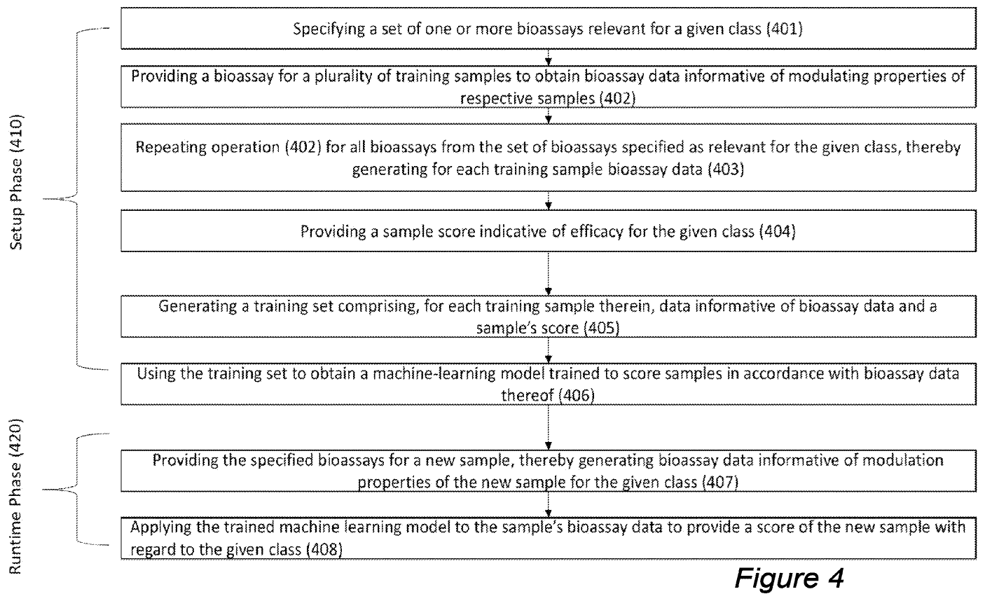

A generalized flow-chart of machine learning-based scoring the samples for a given class is illustrated in FIG. 4. In accordance with certain embodiments of the presently disclosed subject matter, the process includes a setup phase 410 of training a machine learning model to provide scores of the samples and runtime stage 420 of providing scoring of each given new sample. The illustrated scoring can be provided for one or more classes of interest during or after classifying the samples into one or more classes or can be provided alternatively to classifying the samples into a class of interest.

The setup phase can comprise specifying (401) a set of one or more bioassays relevant for the given class, and for each training sample providing a bioassay (from the set of bioassays) to obtain (402) bioassay data informative of modulating properties of the respective training sample for the provided bioassay.

Likewise in the process of FIG. 3, optionally bioassay data can be informative merely of values of attributes defined as “significant” for the provided bioassay. A set of attributes significant for a given bioassay can be predefined by an expert or can be derived from bioassay results with the help of statistical and/or mathematical manipulations.

The method further includes repeating operation (402) for all bioassays specified as relevant for the given class and generating (403) training bioassay data (e.g. a multi-label vector) informative of modulating properties of the respective training sample that are relevant for the given class.

The method further comprises processing (e.g. by scoring module 112) the training bioassay data to provide, for each training sample, a score (404) indicative of efficacy for the given class. Alternatively or additionally, at least part of the samples can be scored with an involvement of an expert.

By way of non-limiting example, the score of a sample can be provided in accordance with relationship between its bioassay data and predefined criteria with regard to certain one or more attributes (e.g. percentage from the predefined best (and/or worst) value, percentage from the best (and/or worst) value among the training samples, percentage of historically best (and/or worst) value, etc.).

Optionally, the score of the sample can be indicative of assigning the sample to a certain quality group. By way of non-limiting example, the samples with scores between 80 and 100 can be assigned to “excellent” group; the samples with scores between 60 and 80 can be assigned to “good” group; the samples with scores between 40 and 60 can be assigned to “neutral” group; and the samples with scores less than 40 can be assigned to “harmful” group.

The setup phase further comprises generating (405) a training set comprising, for each training sample therein, data informative of its bioassay data and a respective sample's score, and using the training set to obtain (406) a machine-learning model trained to score the usability of samples for a given class in accordance with bioassay data thereof.

Efficiency of the training process for classification and/or scoring can be improved by selecting, among the training samples, the training samples for including into the training set.

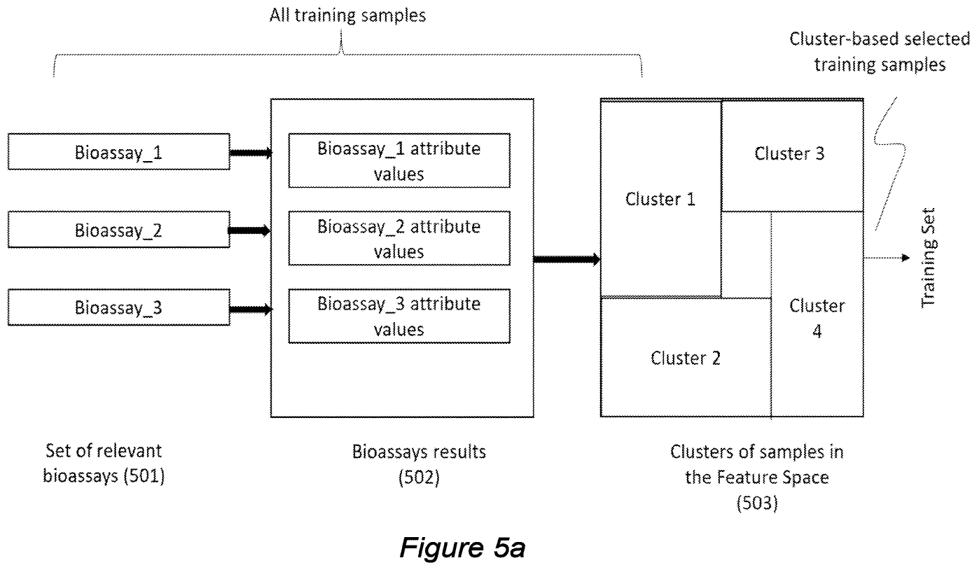

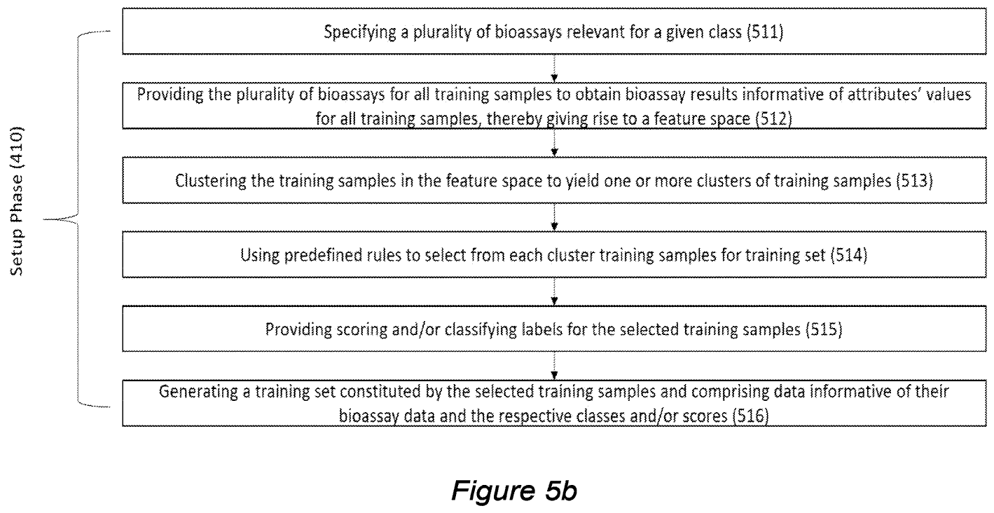

FIGS. 5a and 5b illustrate a schematic diagram and a generalized flow-chart of such “smart” sampling in accordance with certain embodiments of the presently disclosed subject matter.

Like sampling detailed with reference to FIGS. 3 and 4, the smart sampling process includes specifying a plurality of bioassays 501 relevant for a given class (511) and providing the plurality of bioassays for all training samples to obtain (512) bioassay results 502 informative of attributes' values for all training samples and representative by a feature space.

It is noted that for purpose of illustration only, in the following description of “smart” sampling is provided for one class. It will be understood by those skilled in the art that the presently disclosed subject matter is, likewise, applicable to a multi-class training set.

For “smart” sampling, bioassay results 502 are further processed to cluster the training samples in the feature space and yield (513) one or more clusters 503 of training samples. The “smart” sampling further comprises using predefined rules to select (514), from each cluster, training samples for the training set.

The above-detailed “smart” sampling can be further optimized by ranking (e.g. with the help of experts) the relevance of bioassays and/or attributes thereof for the class of interest. Such ranking can be further used for weighting the bioassays results when clustering and/or selecting the training samples from the clusters.

The predefined rules for the cluster-based sample selection can specify, at least, the minimal and/or the maximal numbers of selected training samples (e.g. 10% of the overall training samples but not less than 20 samples), the rules of selection between clusters (e.g. equal number from each cluster or proportionally to the number of samples in the cluster, number weighted in according with provided rating of bioassays/attributes, etc.); the rules of selection within a cluster (randomly, most representative samples, in accordance with distribution of samples in the cluster, etc.).

It is noted that, optionally, the samples for the training set can be selected using predefined rules applied for non-clustered feature space. The predefined rules can specify the minimal and/or the maximal number of selected training samples and the rules of selecting. By way of-non-limiting example, distribution of the selected samples in the feature space can correspond to the distribution of all tested samples in the feature space. Further, the predefined rules can, optionally, consider the rating of bioassays and/or attributes thereof.

The selected training samples are further provided (515) with scoring and/or classification labels and used to generate (516) a training set constituted by the selected training samples and comprising data informative of their bioassay data and respective classes and/or scores.

Referring back to FIG. 4, the runtime phase can comprise providing the specified bioassays for a new sample, thereby generating (407) a multi-label vector informative of values of the informative attributes and applying the trained machine learning model to the multi-label vector to provide (408) a score of the new sample with regard to the given class.

A variety of bioassays are known for determining suitability or effectiveness of a material, i.e., chemical or biological, in treating or preventing a disease or disorder. The bioassays are utilized as analytical tools to determine concentration or potency of the material by its effect in vitro/in vivo. In the great majority of the cases, the bioassays utilized are quantitative assays used to estimate the potency of agents by observing their effects in vitro/in vivo. In bioassaying the extracts or materials, their composition or identity is not known or does not need to be known. Thus, alternatively or additionally to the chemical profiles, the extracts or materials can be catalogued based on their biological activity as reflected from the bioassays.

The bioassay may be any of the bioassays known in the art. These may include: extracellular ATP bioluminescent assay, caspase-1 activity bioluminescent assay, immuneassays, G-CSF immunoassay, GM-CSF immunoassay, HMGB1 immunoassay, immunoassay for specific interleukins, measuring oxidative stress markers, e.g., direct and indirect markers, fluorescent dye-based assays, e.g., oxygen radical antioxidant capacity (ORAC) assay and Trolox equivalent antioxidant capacity (TEAC) assay, quantitative and qualitative analyses of enzymatic reaction products by enzymatic assays and native gel assays, immunohistochemical analyses of tissue and cellular distribution and levels of specific enzymes, urinary prostaglandin E2, quantitative RT-PCR determining relative expression of specific genes in leukocytes and tissues, CT, cytokines, blood bioassay, prostate cancer assay, ovarian cancer assay, breast cancer assay, tumor bioassay, colorectal cancer, and others.

Non-limiting examples of usable bioassays and attributes thereof include:

| Class | | |
|---|---|---|
| (Field of | Exemplified set of bioassays | Attributes usable for |
| treatment) | relevant for the class | bioassays |
| Inflammation | | |
| | Extracellular ATP bioluminescent | Elevated levels of |
| | assay | extracellular ATP release |
| | | from damaged cells |
| | | triggering inflammation |
| | Caspase-1 activity bioluminescent | Altered activity of |
| | assay | Caspase-1, a key |
| | | component of host |
| | | response to microbial |
| | | pathogens and cytokine |
| | | production |
| | G-CSF immunoassay | Levels of Granulocyte |
| | | Colony Stimulating |
| | | Factor (G-CSF), a key |
| | | regulator of the innate |
| | | immune system (NLRP3 |
| | | inflammasome) |
| | GM-CSF immunoassay | Levels of Granulocyte |
| | | and Macrophage Colony |
| | | Stimulating Factor (GM- |
| | | CSF), a regulator of the |
| | | NLRP3 inflammasome |
| | | and cytokine production |
| | HMGB1 immunoassay | Elevated extracellular |
| | | relcase of High Mobility |
| | | Group Box (HMGB1) |
| | | protein from damages |
| | | cells induced inflammation |
| | Immunoassay for specific interleukins | Levels of |
| | | proinflammatory |
| | | cytokines, e.g.: |
| | | Interferon gamma (IFN-γ) |
| | | Interleukin 1 (IL-1) |
| | | Interleukin 1-beta (IL-1β) |
| | | Interleukin 2 (IL-2) |
| | | Interleukin 6 (IL-6) |
| | | Interleukin 10 (IL-10) |
| | | Interleukin 12 (IL-12) |
| | | Interleukin 13 (IL-13) |
| | | Interleukin 15 (IL-15) |
| | | Interleukin 22 (IL-22) |
| | | Interleukin 23 ((IL-23) |
| | Immunoassay | Levels of multifunctional |
| | | cytokine Transforming |
| | | Growth Factor beta |
| | | (TGF-β) |
| | Immunoassay | Levels of |
| | | oroinflammatory |
| | | cytokine Tumour |
| | | Necrosis Factor alpha |
| | | (TNF-α) |
| Oxidative | Measuring oxidative stress markers, | |
| stress | direct and indirectmarkers | |
| | Fluorescent dye-based assays, e.g., | Elevated levels of |
| | Oxygen Radical Antioxidant Capacity | intracellular Reactive |
| | (ORAC) assay and Trolox Equivalent | Oxygen or Nitrogen |
| | Antioxidant Capacity (TEAC) assay. | Species (ROS) and |
| | | (RNS), e.g.: |
| | | peroxides |
| | | superoxides |
| | | hydroxyl radicals |
| | | singlet oxygen |
| | | alpha-oxygen |
| | | peroxynitrite |
| | | nitric oxide |
| | Quantitative and qualitative | Altered levels and/or |
| | analyses of enzymatic reaction | activity of oxidative |
| | products by enzymatic assays and | stress-related enzymes, |
| | native gel assays. | e.g.: |
| | Immunohistochemical analyses of | Superoxide |
| | tissue and cellular distribution and | Dismutases (SOD) |
| | levels of specific enzymes | Glutathione |
| | | Peroxidases (GPX) |
| | | Catalases (CAT) |
| | | Nitric Oxide |
| | | Synthetases (eNOS, |
| | | INOS, nNOS) |
| | | Thioredoxines |
| | Quantitative and qualitative | Indirect measurement of |
| | analyses of enzymatic reaction | oxidative damage, e.g.: |
| | products by enzymatic assays | Ratio of reduced (GSH) |
| | and native gel assays. | and oxidized forms of |
| | Immunohistochemical | glutathione (GSSG) |
| | analyses of tissue and cellular | Intermediates of |
| | distribution and levels of | lipid peroxidation |
| | specific enzymes | Serum albumin redox |
| | | state |
| Pain and | Quantitative and qualitative | Levels and activity of |
| inflammation | analyses of enzymatic reaction | Cyclooxygenase-2 |
| | products by enzymatic assays and | (Cox 2) |
| | native gel assays. | |
| | Immunohistochemical analyses of | |
| | tissue and cellular distribution and | |
| | levels of specific enzymes | |
| | Urinary prostaglandin E2 | Prostaglandins levels |
| | Quantitative RT-PCR determining | Activity of sodium and |
| | relative expression of specific | calcium receptors |
| | genes in leukocytes and tissues | channels, synaptic |
| | | regulators e.g.: |
| | | Vanilloid receptors (TRPV1) |
| Cardiovascular | CT | Chronic stage of CDV |
| disease | | and prognosis, e.g. |
| (CDV) | | coronary calcium |
| | CT | Inflammation (C-reactive |
| | | protein, IL 6, Fibrinogen, |
| | | Monocyte Chemotactic |
| | | Protein-1 (MCP-1), TNF-α |
| | CT | Oxidative stress (e.g., |
| | | isoprostanes) |
| | CT | Metabolic deregulation |
| | | (lipoprotein (a), low- |
| | | density lipoproteins, high |
| | | density lipoprotein, |
| | | ApoB 100, Lipoprotein- |
| | | associated phospholipase |
| | | A2, Homocysteine, |
| | | vitamin D, fibroblast |
| | | growth factor 23, |
| | | adiponectin, glycated |
| | | hemoglobin, haptoglobin etc) |
| Metabolic | | |
| disease | | |
| | Cytokines | Pro-inflammatory |
| | | cytokines (IL-6, IL-10, |
| | | TNF-α) |
| | Cytokines | Markers of pro-oxidant |
| | | status (OxLDL, uric acid) |
| | Cytokines | Prothrombic factors |
| | | (PAI-1) |
| | Cytokines | Other hormones, e.g., |
| | | ghrelin, adiponectin, and |
| | | antioxidant factors |
| | | (PON-1) |
| Neurological | Blood bioassay | Acetylcholine receptor |
| disorders, e.g.: | | antibody |
| muscle weakness | | |
| (Myasthenia | | |
| gravis) | | |
| Cancers | Prostate cancer | PSA, PCA3 |
| | Ovarian Cancer | CA125 |
| | Breast cancer | CA 27-29, CA 15-3, |
| | | CA27. 29, |
| | Tumor bioassay | tissue polypeptide |
| | | specific antigen, p53, |
| | | cathepsin D, cyclin E, |
| | | nestin and HER-2, |
| | | estrogen and progesterone |
| | | receptors ER and PR |
| | Colorectal cancer | carcinoembryonic |
| | | antigen |

The results from each bioassay were compared to the effect of gold standard treatments. The results were validated in an in vivo study, in a respective mice model, assuring liability of the screening methods as a predictive tool. In parallel, data on each of the extracts tested was collected; the data included extract source, major cannabinoid content, and its biological functionality in each of the in vitro bioassays used. Thereafter, the data obtained on each extract was processed and the extract received a score, based on the scoring algorithm disclosed herein.

As stated herein, classification and/or scoring of a sample in accordance with the teachings of the presently disclosed subject matter does not necessarily correlate with expectations based on the chemical properties of the samples. By using the scoring bioassay-based methodology disclosed herein it was determined that many extract combinations traditionally believed to have a specific therapeutic effectiveness towards a particular disease or disorder, due to presence of a particular cannabinoid or a cannabinoid combination in a particular ratio, were in fact ineffective for treating or preventing the particular disease or disorder.

Example 1: two extracts, each containing ˜60% total CBD and ˜2% total THC were expected to have identical therapeutic profiles. However, while one of the extracts was highly effective against ROS, the second was ineffective against ROS.

Example 2: an extract containing 98% CBD was proven to be ineffective as a Cox 2 inhibitor. A different extract containing 99% CBD showed high effectiveness as a Cox 2 inhibitor.

Example 3: an extract containing 56% THC was expected to be highly effective against TNFa and was therefore expected to exhibit anti-inflammatory properties. However, the extract exhibited no anti-inflammatory properties when tested according to the invention.

| | | Attribute | | |
|---|---|---|---|---|
| Sample | Extract | value | Classification (disease) | Comments |
| 1 | 1^{st} extract | High ROS | Anti-inflammatory agent, | Chemical composition |
| | | | reducing oxidative stress, for | identical to 2^{nd} extract- |
| | | | improving cognition | identical attributes are |
| | | | | expected. No correlation |
| | | | | with bioassay |
| | 2^{nd} extract | Low ROS | Inverse to 1^{st} extract | |
| 2 | 1^{st} extract | Cox2 | Pain and inflammation | 98% CBD |
| | | inhibitor | | Chemical composition |
| | | | | identical to 2^{nd} extract, |
| | | | | identical attributes are |
| | | | | expected. No correlation |
| | | | | with bioassay |
| | 2^{nd} extract | Not a Cox2 | Inverse to ^{1st} extract: Pain and | 99% CBD |
| | | inhibitor | inflammation | |
| 3 | | Not | Not effective against TNFa, not | 56% THC |
| | | effective | anti-inflammatory | expected high |
| | | against | | effectiveness against |
| | | TNFa | | TNFa |

As Table 1 shows, no correlation necessary exists between the conclusions derived based on the bioassays and those based on the composition of the extracts. Conclusions made based on the methodology disclosed herein are far more predictive of an actual in vivo therapeutic effect.

It is to be understood that the invention is not limited in its application to the details set forth in the description contained herein or illustrated in the drawings. The invention is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the presently disclosed subject matter.

It will also be understood that the system according to the invention may be, at least partly, implemented on a suitably programmed computer. Likewise, the invention contemplates a computer program being readable by a computer for executing the method of the invention. The invention further contemplates a non-transitory computer-readable memory tangibly embodying a program of instructions executable by the computer for executing the method of the invention.

Those skilled in the art will readily appreciate that various modifications and changes can be applied to the embodiments of the invention as hereinbefore described without departing from its scope, defined in and by the appended claims.

1. A method of computer-based classifying a medical extract from a non-animal eukaryotic source, the method comprising:
for a sample of a non-mammalian eukaryotic extract to be classified, obtaining results of a set of one or more bioassays informative of modulating properties of the sample, thereby generating a bioassay data informative of values of attributes related to the modulating properties; and
applying to the bioassay data a trained machine learning model (MLM) to classify the sample into a class of one or more predefined classes, wherein the MLM is trained to classify samples into the one or more predefined classes in accordance with bioassay data thereof, the training provided with the help of a training set comprising a plurality of training samples, each characterized by a respective bioassay data and a class associated with the sample.

2. The method of claim 1, further comprising classifying the extract in accordance with the class of the respective sample.

3. The method of claim 2, further comprising using the classified extract for at least one of: manufacturing a medical product with treatment properties corresponding to the respective class; standardization of medical extracts in accordance with the one or more classes.

4. The method of claim 1, further comprising screening the extracts to reveal one or more extracts belonging to a given class.

5. The method of claim 1, wherein classifying is provided to define belonging the sample to a given class, the method further comprising predefining a set of bioassays and respective attributes related to the given class.

6. The method of claim 1, further comprising applying to the bioassay data a trained machine learning model (MLM) to obtain a score indicative of efficacy of the sample for the class it has been classified, wherein the MLM is trained to score samples for respective class in accordance with bioassay data thereof, the training provided with the help of a training set comprising a plurality of training samples, each characterized by a respective bioassay data and a score for the respective class.

7. The method of claim 1, wherein generating the training set comprises providing the one or more bioassays to a plurality of training samples to yield a feature space representing the obtained bioassay data; and applying predefined rules to the feature space to select, among the plurality of training samples, training samples to be included in the training set.

8. The method of claim 7, further comprising clustering the samples in the feature space prior to selecting the training samples for the training set, wherein the predefined rules specify, at least, the minimal and/or the maximal numbers of selected training samples in the training set, the rules of selection the samples between the clusters and the rules of selection within the clusters.

9. The method of claim 7, wherein the predefined rules further consider ranking the relevance of bioassays and/or attributes thereof for the respective classes.

10. A computer-based method of providing a medical extract from a non-animal eukaryotic source with a score indicative of efficacy of the extract for a given class, the method comprising:
at a setup phase:
upon specifying one or more bioassays relevant for the given class, a) providing for a plurality of training samples a bioassay among the one or more bioassays to obtain bioassay results informative of modulating properties of the respective samples; repeating operation a) for all the one or more bioassays thereby obtaining bioassay data for the training samples from the plurality of training samples;
generating a training set comprising, for each training sample included therein data informative of its bioassay data and a respective sample's score; and using the training set to obtain a machine-learning model trained to score samples in accordance with bioassay data thereof,
at a runtime phase:
providing the one or more bioassays for a new sample, thereby generating bioassay data informative of modulating properties thereof, and
applying the trained machine learning model to its bioassay data to provide a score of the new sample with regard to the given class.

11. The method of claim 10, wherein generating the training set comprises representing the obtained bioassay data of the plurality of training samples by a feature space; and applying predefined rules to the feature space to select, among the plurality of training samples, training samples to be included in the training set.

12. The method of claim 11, further comprising clustering the samples in the feature space prior to selecting the training samples for the training set, wherein the predefined rules specify, at least, the minimal and/or the maximal numbers of selected training samples in the training set, the rules of selection the samples between the clusters and the rules of selection within the clusters.

13. The method of claim 11, wherein the predefined rules further consider ranking the relevance of bioassays and/or attributes thereof for the respective classes.

14. A set of bioassays usable for classifying a medical extract from a non-animal eukaryotic source in accordance with claim 1.

15. A set of bioassays usable for providing a score for a medical extract from a non-animal eukaryotic source in accordance with claim 11.

16. One or more computing devices comprising processors and memory, the one or more computing devices configured, via computer-executable instructions, to perform operations for operating, in a cloud computing environment, in accordance with a claim 1.

17. A classification system for classifying and/or scoring a medical extract in accordance with claim 1.

18. A non-transitory computer-readable medium comprising instructions that, when executed by a computing system comprising a memory storing a plurality of program components executable by the computing system, cause the computing system to operate in accordance with claim 1.
